(19) United States
(12) Patent Application Publication
(10) Pub. No.: US 2020/0205834 A1
(43) Pub. Date: Jul. 2, 2020

(54) END-TO-END ANASTOMOSIS INSTRUMENT AND METHOD

(71) Applicant: Covidien LP, Mansfield, MA (US)

(72) Inventor: Thomas E. Drochner, Longmont, CO (US)

(21) Appl. No.: 16/722,134

(22) Filed: Dec. 20, 2019

Related U.S. Application Data
(60) Provisional application No. 62/787,467, filed on Jan. 2, 2019.

(51) Int. Cl. A61B 17/11

(52) U.S. Cl. CPC A61B 17/11; A61B 2017/1132; A61B 2017/1125; A61B 2017/1107

(57) ABSTRACT

A surgical instrument for forming an end-to-end anastomosis includes first and second handles each supporting a shaft at a distal end thereof. The proximal ends of each shaft are coupled about a pivot such that movement of the handles correspondingly moves the distal ends of each shaft. A release mechanism is included that is actuatable to pivot a pair of bifurcated legs of each shaft between a first position for receiving first and second vessels and a second position that facilitate release of the respective first and second vessels. First and second posts are supported on each distal end and are configured to support an end of the respective first and second vessels thereon. The posts are adapted to connect to an electrical generator for communicating energy through each end of each respective first and second vessel to form an anastomotic seal.

### CROSS REFERENCE TO RELATED APPLICATIONS

This application claims the benefit of U.S. Provisional Patent Application Ser. No. 62/787,467 filed Jan. 2, 2019, the entire contents of which are incorporated by reference herein.

### TECHNICAL FIELD

The present disclosure relates to a surgical instrument and method for performing anastomosis of tubular body structures, and more particularly to an instrument for performing an end-to-end anastomosis of two tubular vessels.

### BACKGROUND OF RELATED ART

Anastomoses of tubular body structures may be performed for a number of different procedures. One general example of an anastomosis is a vascular anastomosis wherein two blood vessels are joined together to permit blood flow therebetween. A specific example of vascular anastomosis is an arteriovenous fistula (“A-V fistula”) which is performed to facilitate hemodialysis for end stage kidney disease. The procedure usually consists of an end-to-side anastomosis joining an artery and a vein in the forearm, e.g., joining the radial artery end to side with the cephalic (radial) vein or the ulnar artery with the basilic (ulnar) vein.

Another specific example of a vascular anastomosis is an end-to-end anastomosis. Surgical stapling instruments for applying an annular array of staples or fasteners to tissue are well known in the art for performing end-to-end anastomosis. For example, surgical stapling instruments for applying an annular array of staples, as well as devices for completing a surgical anastomosis through the provision of anastomosis rings, are well known in gastric and esophageal surgery. In classic or modified gastric reconstruction an end-to-end, end-to-side, or side-to-side anastomosis are typically formed with a stapling instrument. These instruments generally include a circular array of fasteners such as staples, anastomosis rings, and the like, while the anvil member includes means for completing the circular anastomosis, typically an array of bucket members that cinch the staples after the staples are expelled from the fastener assembly, or may include a locking member for the anastomosis ring.

Other end-to-end anastomosis techniques involve suturing the two ends of the vessels together. Several techniques have been successfully used in the past as described in Principles of Vascular Anastomosis by Abdulsalam Y Taha (October 2015). The process of manually suturing two vessels in and end-to-end fashion is time consuming and requires a great deal of skill on the part of the surgeon. The resulting success of the sutured anastomosis will also be dependent on the skill of the surgeon. In minimally invasive procedures such as in Minimally Invasive Direct Coronary Artery Bypass (MIDCAB), the ability to suture is even more complicated due to limited maneuverability and reduced visibility.

While the previously mentioned instrument and various techniques use mechanical or manual methods to connect two vessels in an end-to-end manner, there exists a need for improved surgical instrumentation and methods for performing end-to-end anastomosis in a simplified and time efficient manner.

### SUMMARY

Aspects according to the present disclosure relate to a surgical instrument for forming an end-to-end anastomosis and includes first and second handles each supporting a shaft at a distal end thereof. Each shaft includes a proximal end and a distal end. The proximal ends of each shaft are coupled about a first pivot such that movement of the first and second handles correspondingly moves the distal ends of each shaft between a spaced apart position and a position for approximating first and second vessels. Each distal end of each shaft includes bifurcated legs configured to rotate relative to one another about a second pivot. A release mechanism is included having first and second housings coupled to each respective shaft. The release mechanism is actuatable to pivot the bifurcated legs of each shaft between a first position wherein the bifurcated legs of each shaft define an aperture for receiving a respective one of the first and second vessels and a second position wherein the bifurcated legs of each shaft are spaced relative to one another to facilitate release of the respective first and second vessels.

First and second posts are supported on each distal end of each shaft in opposed relation relative to one another. Each post is configured to support an end of the respective first and second vessels thereon and each post is correspondingly bifurcated atop each bifurcated distal leg. The first post is adapted to connect to a first polarity of an electrical generator and the second post is adapted to connect to a second polarity of the electrical generator such that, upon activation of the generator, electrical energy is communicated through each end of each respective first and second vessel to form an anastomotic seal.

In aspects according to the present disclosure each of the posts includes a plurality of stays for securing the end of each respective first and second vessel thereon. In other aspects according to the present disclosure, the release mechanism includes one or more tabs configured to pivot the respective pair of bifurcated legs of each distal end of each shaft. In other aspects, the release mechanism includes two tabs each configured to pivot one of the respective pair of bifurcated legs of each shaft.

In yet other aspects according to the present disclosure, the release mechanism includes a tube disposed in each of the first and second housings and a tab operably disposed on each of the first and second housings, each tab, upon actuation thereof, is configured to translate one of the respective tubes to pivot the respective pair of bifurcated legs of each distal end of each shaft. In aspects, each tube is biased by a spring.

In still other aspects according to the present disclosure, when the first and second vessels are approximated, the plurality of stays with respect to each post creates a gap between posts within the range of about 0.001 inches to about 0.006 inches. In aspects, energy is delivered from the electrical generator utilizing a sealing algorithm to create a seal between the ends of the first and second vessels.

In still other aspects according to the present disclosure, actuation of the first and second handles moves the distal ends of each shaft member in a first direction and actuation of the release mechanism separates the pair of bifurcated legs of each distal end of each shaft member in a second direction that is transverse to the first direction.

Aspects of the present disclosure also relate to a method for performing an end-to-end anastomosis of first and second vessels and includes inserting an end of a first vessel through an aperture defined in a distal end of a shaft of a first handle, the distal end of the first handle bifurcated to form a pair of first and second legs and inserting an end of a second vessel through an aperture defined in a distal end of a shaft of a second handle, the distal end of the second handle bifurcated to form a pair of first and second legs.

The method also includes: everting the ends of the first and second vessels over posts supported on the respective distal ends of the shafts; moving the first and second handles in a first direction to approximate the distal ends of the shafts such that the ends of the first and second vessels abut one another; energizing the posts of the distal ends such that electrical energy is communicated through the ends of the first and second vessels to form an anastomotic seal; moving the first and second handles in a second, opposite direction to space the distal ends of the shafts relative to one another; actuating a release mechanism to separate the pair of first and second legs of each distal end relative to one another; and removing the anastomosed first and second vessels from the respective distal ends of the shafts of the first and second handles.

In aspects according to the present disclosure, movement of the first and second handles moves the distal ends of the shafts in a first direction and actuation of the release mechanism moves the first and second legs of each distal end in a second direction, the second direction transverse to the first direction.

In other aspects according to the present disclosure energizing the posts includes utilizing a sealing algorithm to seal the first and second vessels to create the anastomosis. In other aspects according to the present disclosure the method for performing an end-to-end anastomosis further includes securing the everted ends of the first and second vessels atop a plurality of stays that extend from each post supported on each distal end of each shaft. In still other aspects according to the present disclosure, actuating the release mechanism to separate the pair of first and second legs of each distal end includes actuating a pair of finger tabs to reciprocate a pair of tubes each disposed within each shaft to separate the pair of first and second legs of each distal end.

### BRIEF DESCRIPTION OF THE DRAWINGS

An illustrative embodiment of the subject surgical instrument and method are described herein with reference to the drawings wherein:

FIG. 1 is a perspective view of a surgical instrument for performing an end-to-end anastomosis in accordance with one embodiment of the present disclosure;

FIG. 2A is an enlarged, perspective view of the surgical instrument of FIG. 1 showing and end of a tubular vessel being introduced into a distal end of the surgical instrument;

FIG. 2B is an enlarged, perspective view of the surgical instrument of FIG. 1 showing the end of the tubular vessel being everted over the distal end of the surgical instrument;

FIG. 3A is an enlarged, perspective view of the distal end of the surgical instrument of FIG. 1 showing two opposing ends of two tubular vessels prior to the application of radiofrequency (RF) energy;

FIG. 3B is a, perspective view of the surgical instrument of FIG. 1 showing the two opposing ends of two tubular vessels prior to the application of RF energy;

FIG. 4 is an enlarged, perspective view of the surgical instrument of FIG. 1 showing the two opposing ends of two tubular vessels after the application of RF energy and the formation of an anastomotic seal;

FIG. 5 is an end perspective view of the distal end of the surgical instrument of FIG. 1 showing movement of a release mechanism configured to release the completed anastomosis from the surgical instrument after application of RF energy; and

FIG. 6 is a side perspective view showing the surgical instrument after release of the two vessels and showing the completed anastomosis.

### DETAILED DESCRIPTION

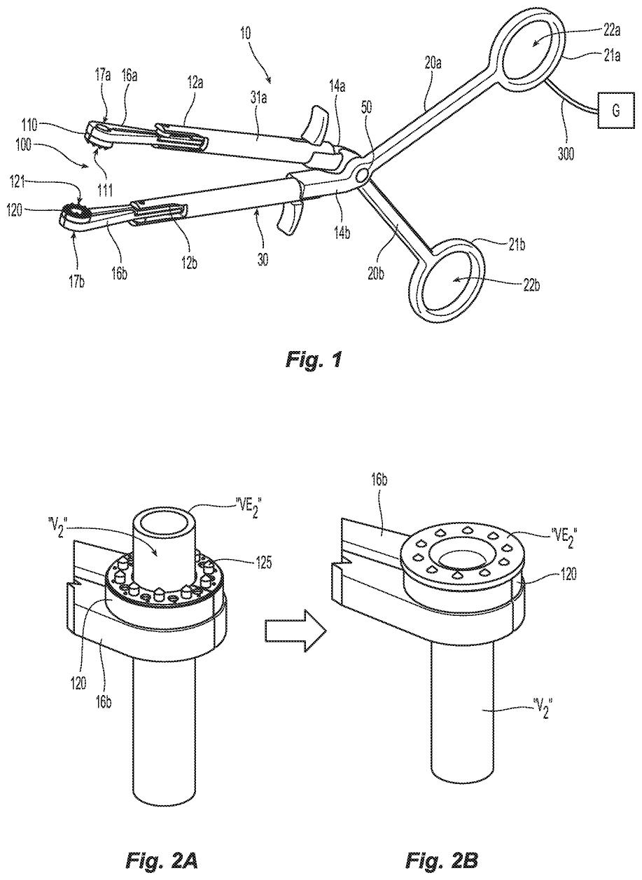

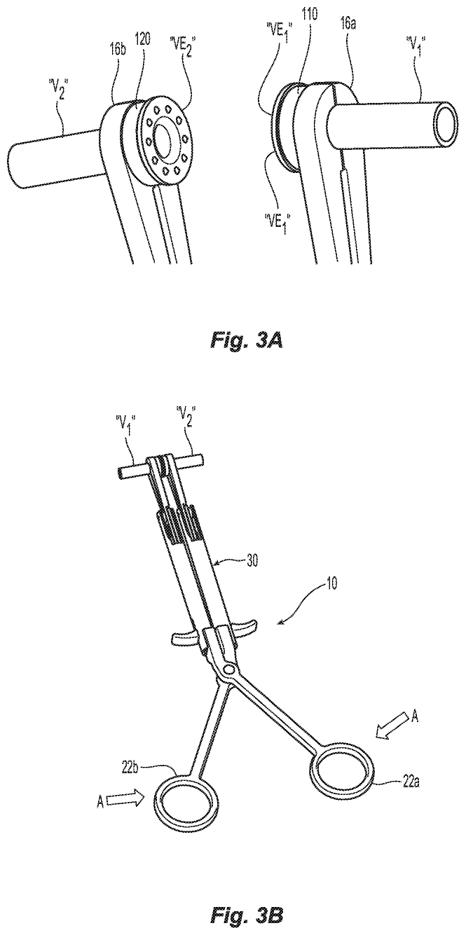

Referring now in detail to the drawing figures in which like reference numerals identify similar or identical elements, one embodiment of the present disclosure is illustrated generally in FIG. 1 and is designated herein as surgical instrument 10. Briefly, surgical instrument 10 includes first and second handles 20a, 20b, respectively, that are each configured to support a ring 21a, 21b at a proximal end thereof which each define a respective finger hole 22a, 22b for receiving a surgeon's finger. Handles 20a, 20b include respective shafts 12a, 12b that extend therefrom and that are joined about a pivot 50 which is configured to allow shafts 12a, 12b to move relative to one another via actuation of handles 20a, 20b between a first, spaced apart position to a second, closer positon for approximating tissue (FIG. 3B).

Shafts 12a, 12b include respective proximal ends 14a, 14b and distal ends 16a, 16b. The distal ends 16a, 16b are configured to support corresponding posts 110, 120, respectively, thereon which, in turn, are configured to secure an end of a vessel thereon as explained in more detail below with reference to FIGS. 2A and 2B. Each distal end 16a, 16b of each shaft 12a, 12b defines an aperture 17a, 17b therethrough that vertically registers with a corresponding aperture 111, 121 defined in each post 110, 120. The two sets of apertures 17a, 111 and 17b, 121 are sized to receive a tubular vessel, vessel V2 (FIGS. 2A and 2B) therethrough.

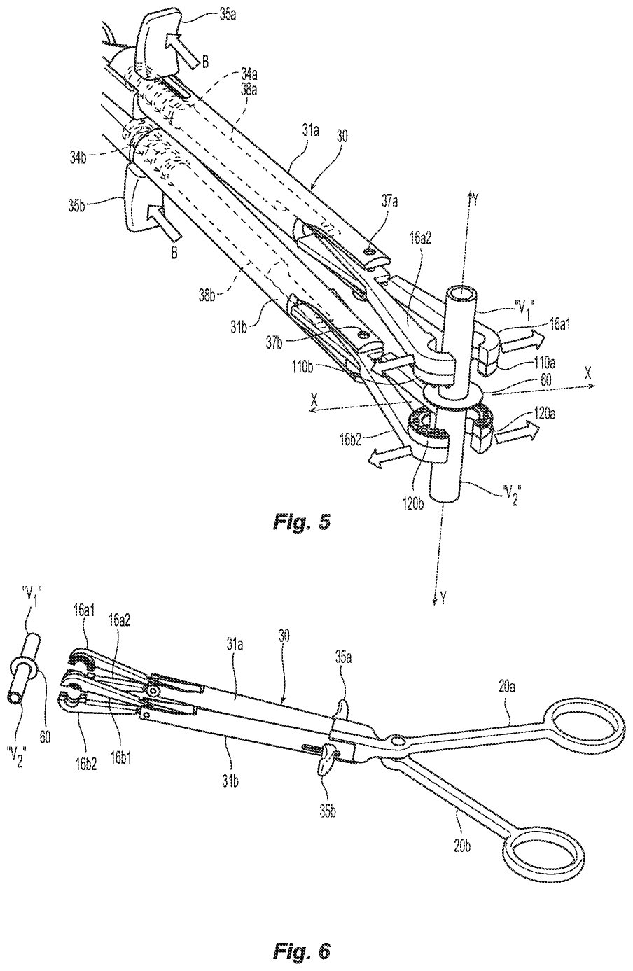

Each distal end 16a, 16b of each shaft 12a, 12b is bifurcated (e.g., includes two legs 16a1, 16a2 and 16b1, 16b2) along a portion of a length thereof which enables a release mechanism 30, upon actuation thereof, to spread each distal end 16a, 16b apart as explained in detail below with reference to FIGS. 5 and 6. Each post 110, 120 is likewise bifurcated such that each bifurcated half 110a, 110b and 120a, 120b is supported by a corresponding leg 16a1, 16a2 and 16b1, 16b2.

As shown in FIGS. 1, 5 and 6, release mechanism 30 includes first and second housings 31a, 31b which are each configured to house a respective shaft 12a, 12b therein and support a respective actuation tab 35a, 35b thereon. The distal end of each housing 31a, 31b supports a pivot 37a, 37b that is configured to engage corresponding distal ends 16a, 16b. Actuation of each tab 35a, 35b translates a respective inner tube 38a, 38b disposed within each housing 31a, 31b against a biasing spring 34a, 34b which, in turn, pivots each bifurcated distal end 16a, 16b to an open configuration thereby spreading legs 16a1, 16a2 and 16b1, 16b2 and allowing the release of the anastomosis from therein (See FIG. 6).

In operation, and as shown initially with respect to FIGS. 2A and 2B, a first vessel, e.g., “V2” is inserted into the aperture 17b defined within distal end 16b and through the aperture 121 defined within post 120 such that the distal end “VE2” extends a length relative thereto (FIG. 2A). The surgeon then everts the distal end “VE2” over the post 120 such that a series of stays 125 (configured to extend from post 120) secure the distal end “VE2” atop the post 120. The same procedure is repeated with respect to a second vessel V1 over post 110.

Once both vessels “V1” and “V2” are properly seated atop respective posts 110 and 120 (FIG. 3A), the handles 20a, 20b are actuated in the direction “A” to approximate the distal ends 16a, 16b and the two vessels “V1”, “V2” next to one another along an axis Y-Y defined through the first and second vessels “V1”, “V2” (See FIG. 5). RF energy is then supplied to posts 110 and 120 to form a fusion, e.g., seal 60 between everted ends “VE1” and “VE2”. A generator “G” is connected via cable 300 to one or both handles 20a, 20b and is configured to supply RF energy to posts 110 and 120 upon selective activation thereof. Other energy modalities are also contemplated that may be utilized to create a fusion of the ends “VE1”, “VE2” of the vessels “V1”, “V2”, ultrasonic energy, microwave energy, light energy, etc.

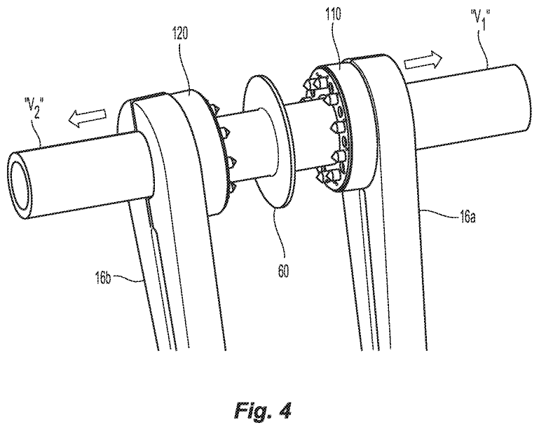

In one contemplated embodiment the generator “G” is configured to utilize a sealing algorithm that supplies electrosurgical energy in a specific manner to create a seal 60 between the two vessel ends “VE1”, “VE2” (FIG. 4). Various sealing algorithms are disclosed in U.S. Pat. Nos. 8,920,421, 8,147,485, 8,216,233, 7,972,328, 7,303,557, 6,796,981 all owned by Covidien AG (a division of Medtronic), the contents of each of which being incorporated by reference herein. In this instance, the stays 125 may be configured to act as stop members to create a gap between posts 110, 120 which is known to promote better quality seals 60. In embodiments, the gap between the posts 110, 120 is in the range of about 0.001 inches to about 0.006 inches. For the purposes herein, the anastomotic fusion that is formed between vessel ends “VE1” and “VE2” will be called seal 60.

Once the seal 60 is formed and the anastomosis between vessel “V1”, “V2” is complete, the handles 20a, 20b are moved back towards the spaced apart position such that the distal ends 16a, 16b slide proximally along each respective vessel “V1”, “V2” and become spaced from the seal 60 (FIG. 4). The surgeon then actuates the release mechanism 30 by pulling back on tabs 35a, 35b in a proximal direction “B” to translate inner tubes 38a, 38b (disposed in housings 31a, 31b). The translation of tubes 38a, 38b pivot each leg 16a1, 16a2 and 16b1, 16b2 of each respective distal end 16a, 16b to an open or spaced-apart positon to facilitate release of each vessel “V1”, “V2” (FIGS. 5 and 6). Once the vessel is released, the bias of springs 34a, 34b return the legs 16a1, 16a2 and 16b1, 16b2 back to a closed position.

As shown in FIGS. 5 and 6, actuation of the handles 20a and 20b move the distal ends in a direction along the axis Y-Y to approximate the distal ends 16a, 16b. Actuation of the release mechanism 30 moves each bifurcated distal end 16a1, 16a2 and 16b1, 16b2 along an axis X-X which is transverse to axis Y-Y.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

For a detailed description of exemplary medical work stations and/or components thereof, reference may be made to U.S. Patent Application Publication No. 2012/0116416, and PCT Application Publication No. WO2016/025132, the entire contents of each of which are incorporated by reference herein.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure.

Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A surgical instrument for forming an end-to-end anastomosis, comprising:
first and second handles each supporting a shaft at a distal end thereof, each shaft including a proximal end and a distal end, the proximal ends of each shaft coupled about a first pivot such that movement of the first and second handles correspondingly moves the distal ends of each shaft between a spaced apart position and a position for approximating first and second vessels, each distal end of each shaft including a pair of bifurcated legs configured to rotate relative to one another about a second pivot;
a release mechanism including first and second housings coupled to each respective shaft, the release mechanism actuatable to pivot the pair of bifurcated legs of each shaft between a first position wherein the pair of bifurcated legs of each shaft define an aperture for receiving a respective one of the first and second vessels and a second position wherein the pair of bifurcated legs of each shaft are spaced relative to one another to facilitate release of the respective first and second vessels; and
first and second posts supported on each distal end of each shaft in opposed relation relative to one another, each post configured to support an end of the respective first and second vessels thereon and each post being correspondingly bifurcated atop each pair of bifurcated legs of each distal end, the first post adapted to connect to a first polarity of an electrical generator and the second post adapted to connect to a second polarity of the electrical generator such that, upon activation of the generator, electrical energy is communicated through each end of each respective first and second vessel to form an anastomotic seal.

2. The surgical instrument according to claim 1 wherein each of the posts includes a plurality of stays for securing the end of each respective first and second vessel thereon.

3. The surgical instrument according to claim 1 wherein the release mechanism includes at least one tab configured to pivot the respective pair of bifurcated legs of each distal end of each shaft.

4. The surgical instrument according to claim 1 wherein the release mechanism includes two tabs each configured to pivot one of the respective pair of bifurcated legs of each shaft.

5. The surgical instrument according to claim 1 wherein the release mechanism includes a tube disposed in each of the first and second housings and a tab operably disposed on each of the first and second housings, each tab, upon actuation thereof, configured to translate one of the respective tubes to pivot the respective pair of bifurcated legs of each distal end of each shaft.

6. The surgical instrument according to claim 5 wherein each tube is biased by a spring.

7. The surgical instrument according to claim 2 wherein, when the first and second vessels are approximated, the plurality of stays with respect to each post creates a gap between posts within the range of about 0.001 inches to about 0.006 inches.

8. The surgical instrument according to claim 7 wherein energy is delivered from the electrical generator utilizing a sealing algorithm to create a seal between the ends of the first and second vessels.

9. The surgical instrument according to claim 1 wherein actuation of the first and second handles moves the distal ends of each shaft member in a first direction and actuation of the release mechanism separates the pair of bifurcated legs of each distal end of each shaft member in a second direction that is transverse to the first direction.

10. A method for performing an end-to-end anastomosis of first and second vessels, comprising:
inserting an end of a first vessel through an aperture defined in a distal end of a shaft of a first handle, the distal end of the first handle bifurcated to form a pair of first and second legs;
inserting an end of a second vessel through an aperture defined in a distal end of a shaft of a second handle, the distal end of the second handle bifurcated to form a pair of first and second legs;
everting the ends of the first and second vessels over posts supported on the respective distal ends of the shafts;
moving the first and second handles in a first direction to approximate the distal ends of the shafts such that the ends of the first and second vessels abut one another;
energizing the posts of the distal ends such that electrical energy is communicated through the ends of the first and second vessels to form an anastomotic seal;
moving the first and second handles in a second, opposite direction to space the distal ends of the shafts relative to one another;
actuating a release mechanism to separate the pair of first and second legs of each distal end relative to one another; and
removing the anastomosed first and second vessels from the respective distal ends of the shafts of the first and second handles.

11. The method for performing an end-to-end anastomosis according to claim 10 wherein movement of the first and second handles moves the distal ends of the shafts in a first direction and actuation of the release mechanism moves the first and second legs of each distal end in a second direction, the second direction transverse to the first direction.

12. The method for performing an end-to-end anastomosis according to claim 10 wherein energizing the posts includes utilizing a sealing algorithm to seal the first and second vessels to create the anastomosis.

13. The method for performing an end-to-end anastomosis according to claim 10 further comprising securing the everted ends of the first and second vessels atop a plurality of stays that extend from each post supported on each distal end of each shaft.

14. The method for performing an end-to-end anastomosis according to claim 10 wherein actuating the release mechanism to separate the pair of first and second legs of each distal end includes actuating a pair of finger tabs to reciprocate a pair of tubes each disposed within each shaft to separate the pair of first and second legs of each distal end.
